SEQUENCE LISTING (56 sequences)

SEQ ID NO: 1 - protein, 10 aa, JC polyomavirus
MAGVAWIHCL

SEQ ID NO: 2 - protein, 10 aa, JC polyomavirus
FLRSSPLVWI

SEQ ID NO: 3 - protein, 9 aa, JC polyomavirus
ILMWEAVTL

SEQ ID NO: 4 - protein, 10 aa, JC polyomavirus
STISQAFWNL

SEQ ID NO: 5 - protein, 10 aa, JC polyomavirus
RILIFLLEFL

SEQ ID NO: 6 - protein, 10 aa, JC polyomavirus
FESDSPNRDM

SEQ ID NO: 7 - protein, 10 aa, JC polyomavirus
YPISFLLTDL

SEQ ID NO: 8 - protein, 10 aa, JC polyomavirus
SAIAQLGYRF

SEQ ID NO: 9 - protein, 9 aa, JC polyomavirus
LTCGNILMW

SEQ ID NO: 10 - protein, 9 aa, JC polyomavirus
GEAAATIEV

SEQ ID NO: 11 - protein, 10 aa, JC polyomavirus
RILIFLLEFL

SEQ ID NO: 12 - protein, 9 aa, JC polyomavirus
FPVDLHAFL

SEQ ID NO: 13 - protein, 9 aa, JC polyomavirus
GFGGHNILF

SEQ ID NO: 14 - protein, 9 aa, JC polyomavirus
VSWKLVTQY

SEQ ID NO: 15 - protein, 9 aa, JC polyomavirus
LLMGMYLDF

SEQ ID NO: 16 - protein, 10 aa, JC polyomavirus
FLLDKMDLIF

SEQ ID NO: 17 - protein, 10 aa, JC polyomavirus
MAGVAWIHCL

SEQ ID NO: 18 - protein, 9 aa, JC polyomavirus
IPKKRYWLF

SEQ ID NO: 19 - protein, 10 aa, JC polyomavirus
ISMYTFSTMK

SEQ ID NO: 20 - protein, 10 aa, JC polyomavirus
WPNCATNPSV

SEQ ID NO: 21 - protein, 10 aa, JC polyomavirus
FLRSSPLVWI

SEQ ID NO: 22 - protein, 10 aa, JC polyomavirus
FESDSPNRDM

SEQ ID NO: 23 - protein, 9 aa, JC polyomavirus
LTCGNILMW

SEQ ID NO: 24 - protein, 9 aa, JC polyomavirus
ILMWEAVTL

SEQ ID NO: 25 - protein, 10 aa, JC polyomavirus
ILMWEAVTLK

SEQ ID NO: 26 - protein, 9 aa, JC polyomavirus
YPDGTIFPK

SEQ ID NO: 27 - protein, 10 aa, JC polyomavirus
YPISFLLTDL

SEQ ID NO: 28 - protein, 9 aa, JC polyomavirus
TPRVDGQPM

SEQ ID NO: 29 - protein, 9 aa, JC polyomavirus
GEAAATIEV

SEQ ID NO: 30 - protein, 9 aa, JC polyomavirus
VEIASLATV

SEQ ID NO: 31 - protein, 9 aa, JC polyomavirus
MALQLFNPE

SEQ ID NO: 32 - protein, 10 aa, JC polyomavirus
STISQAFWNL

SEQ ID NO: 33 - protein, 10 aa, JC polyomavirus
RFLEETTWAI

SEQ ID NO: 34 - protein, 9 aa, BK polyomavirus
RIFIFILEL

SEQ ID NO: 35 - protein, 9 aa, BK polyomavirus
FPSDLHQFL

SEQ ID NO: 36 - protein, 9 aa, BK polyomavirus
MCAGHNIIF

SEQ ID NO: 37 - protein, 9 aa, BK polyomavirus
VSWKLITEY

SEQ ID NO: 38 - protein, 9 aa, BK polyomavirus
LLLGMYLEF

SEQ ID NO: 39 - protein, 10 aa, BK polyomavirus
HILDKMDLIF

SEQ ID NO: 40 - protein, 10 aa, BK polyomavirus
MAGVAWILCL

SEQ ID NO: 41 - protein, 9 aa, BK polyomavirus
VPKRRYWLF

SEQ ID NO: 42 - protein, 10 aa, BK polyomavirus
ISMYTFSTRK

SEQ ID NO: 43 - protein, 10 aa, BK polyomavirus
WPICSKKPSV

SEQ ID NO: 44 - protein, 10 aa, BK polyomavirus
FLRKEPLVWI

SEQ ID NO: 45 - protein, 10 aa, BK polyomavirus
FSSDSPERKM

SEQ ID NO: 46 - protein, 9 aa, BK polyomavirus
LTCGNLLMW

SEQ ID NO: 47 - protein, 9 aa, BK polyomavirus
LLMWEAVTV

SEQ ID NO: 48 - protein, 10 aa, BK polyomavirus
LLMWEAVTVQ

SEQ ID NO: 49 - protein, 9 aa, BK polyomavirus
YPDGTITPK

SEQ ID NO: 50 - protein, 10 aa, BK polyomavirus
YPISFLLSDL

SEQ ID NO: 51 - protein, 9 aa, BK polyomavirus
TQRVDGQPM

SEQ ID NO: 52 - protein, 9 aa, BK polyomavirus
GEAAAAIEV

SEQ ID NO: 53 - protein, 9 aa, BK polyomavirus
VQIASLATV

SEQ ID NO: 54 - protein, 9 aa, BK polyomavirus
MALELFNPD

SEQ ID NO: 55 - protein, 10 aa, BK polyomavirus
ATISQALWHV

SEQ ID NO: 56 - protein, 10 aa, BK polyomavirus
RFLEETTWTI

(19) United States
(12) Patent Application Publication
(10) Pub. No.: US 2017/0059568 A1
(43) Pub. Date: Mar. 2, 2017

(54) Identification of JC viral epitopes by cytotoxic T lymphocytes in healthy individuals and their use in assays

(71) Applicant: JANSSEN PHARMACEUTICA NV, Beerse (BE)

(72) Inventor: Lieven Jozef Stuyver, Herzele (BE)

(21) Appl. No.: 15/119,793

(22) PCT Filed: Feb. 19, 2015
(86) PCT No.: PCT/EP2015/053460

§ 371 Date: Aug. 18, 2016

(30) Foreign Application Priority Data
Feb. 20, 2014 (EP) 14155984.9

(51) Int. Cl. G01N 33/569

(52) U.S. Cl. CPC G01N 33/56983; G01N 2800/50; G01N 2333/025; G01N 2800/285

(57) ABSTRACT

The invention relates to a method to predict the onset and/or development of PML in a patient or in an immunosuppressed patient by detection of JCV specific CTL's in blood of said patient using any of the ten (10) JCV amino acid sequences SEQ ID NO 1-10 separately or in combination with each other.

The detection of JCPyV (JC polyomavirus) reactive cytotoxic T lymphocytes in peripheral blood has been associated with a favorable outcome in patients with progressive multifocal leucoencephalopathy. The frequency of these cells in peripheral blood mononuclear cells of healthy volunteers is unknown. To help the development of a highly sensitive assay for detecting cellular immune responses against JC virus, according to the current invention a CTL prediction and mapping study of the whole JCPyV genome has been performed. A total of 98 peptides were selected, and subsequently tested in the 10 most common allotypes. Immune responses were evaluated in an optimized direct ex vivo ELISPOT assay, using 5*10^{5 CD}8+-enriched peripheral blood mononuclear cells per well. A novel panel of 10 immunodominant epitopes was defined of which 75 to 100% were recognized in a cohort of 20 healthy controls representing the most common allotypes. Some of these novel epitopes were used to illustrate a polyomavirus-specific T cell response in otherwise JCPyV antibody-negative individuals. The newly discovered epitopes were located in T antigen, VP1, VP2, and agnoprotein. No significant immune responses against the predicted epitopes were identified for B*0702 and B*3501 allotypes.

Cytotoxic T cell (CTL) responses play a crucial role in the control of many viral infections in humans. Cytotoxic T-lymphocyte is a T cell that is antigen-specific and is able to search out and kill specific types of virus-infected cells. When cytotoxic T-lymphocytes (CTLs) find cells carrying the viral peptide they are looking for, they induce these cells to secrete proteins that attract nearby macrophages (a type of white blood cells). These macrophages then surround and destroy the infected cells. CTLs expresses the CD8 transmembrane glycoprotein and are therefore also known as CD8+ T cells or CT cells. CTLs recognize virus-infected cells through the interaction of T-cell receptor (TCR) with 8-10 amino acid (aa) viral epitopes in association with an MHC class I molecule on the surface of infected cells. There is a high medical need to characterize the cellular immune response against the human JC polyomavirus (JCPyV), the etiological agent of progressive multifocal leukoencephalopathy (PML).

JCPyV is a polyomavirus that infects the vast majority of the adult population worldwide. Renal tubular epithelial cells, the urinary tract and lymphoid organs are sites of viral latency in most infected individuals of which approximately 30% shed JCPyV DNA in the urine. The mechanisms controlling JCPyV replication and latency in normal individuals are not well understood.

PML is a demyelinating disease of the central nervous system (CNS) caused by reactivation of JCPyV in the setting of immunosuppression. Although IgG antibodies (Abs) specific for JCPyV can be detected in up to 50-60% of the normal adult population, this humoral response is unable to prevent the development of PML. This disease occurs in patients with AIDS, cancer, in organ transplant and in 0.1% of multiple sclerosis (MS) patients receiving natalizumab.

Since JCPyV reactivation occurs as a consequence of immune suppression, and humoral immunity does not appear to be a key player in controlling JCPyV virus replication, cell-mediated immunity may play a more prominent role. In previous studies it has been demonstrated that the detection of CD8+ CTLs against A*0201 restricted JCPyV epitopes VP1p36 and VP1p100 after peptide stimulation and cultivation for 10-14 days was associated with better outcome for PML patients. JCPyV-specific CTLs were also detected in the blood of most healthy volunteers after in vitro stimulation, suggesting that these cells might contribute to controlling viral replication and the protection against PML in immunocompetent people.

Frequencies of JCPyV-specific CTLs are estimated to be very low in fresh blood. The assays used until now are a Cr release assay, which is limited by the use of radioactivity and the difficulty to label target cells with ⁵¹Cr and a tetramer staining, which is limited by HLA restriction of the selected epitope.

There is a high medical need for a method to predict that patients suffering from for instance MS (multiple sclerosis) when treated with a drug, like natalizumab, will whether or not potentially develop PML by the detection of JCPyV-specific CTLs in the blood of the patient.

The current invention relates a method or assay, a so-called direct ex-vivo ELISPOT, measuring JCPyV specific T-cells. Previously identified HLA class I T cell epitopes were initially used as test antigens for the optimization of the IFN-gamma T cell responses by ELISPOT. The frequency of T cell responses to these epitopes has previously been reported to be low compared to other viruses such as cytomegalovirus and Epstein Barr virus and it has been shown that enrichment of CD8+ T cells in the PBMC fraction can enhance the detection of specific T cell responses to JCPyV HLA class I peptides. Besides this, an in-silico algorithm was applied to predict epitopes for JCPyV and BKPyV. A selection of these predicted epitopes was tested in the optimized ex-vivo ELISPOT assay in 92 healthy subjects with different HLA-haplotypes, to experimentally define new frequently recognized epitopes. Ten (10) epitopes of JCPyV have been identified and form part of the invention. Confirmation of these reactive peptides was obtained in a cohort of 20 healthy subjects.

The present invention aims to the use of any of the ten (10) JCV amino acid sequences SEQ ID NO 1-10 separately or in combination with each other to detect JCV specific CTL's in blood of a patient or in an immunosuppressed patient to predict the onset and/or development of PML.

Part of the invention is a method to predict the onset and/or development of PML in a patient or in an immunosuppressed patient by detection of JCV specific CTL's in blood of said patient using any of the ten (10) JCV amino acid sequences SEQ ID NO 1-10 separately or in combination with each other.

SEQ ID NO 1-10 is as follows:

SEQ ID NO 1:
MAGVAWIHCL
SEQ ID NO 2:
FLRSSPLVWI
SEQ ID NO 3:
ILMWEAVTL
SEQ ID NO 4:
STISQAFWNL
SEQ ID NO 5:
RILIFLLEFL
SEQ ID NO 6:
FESDSPNRDM
SEQ ID NO 7:
YPISFLLTDL
SEQ ID NO 8:
SAIAQLGYRF
SEQ ID NO 9:
LTCGNILMW
SEQ ID NO 10:
GEAAATIEV

ELISPOT Assay Principle

ELISPOT assays employ the sandwich enzyme-linked immunosorbent assay (ELISA) technique. Either a monoclonal or polyclonal antibody specific for the chosen analyte is pre-coated onto a PVDF (polyvinylidene difluoride)-backed microplate. Appropriately stimulated cells are pipetted into the wells and the microplate is placed into a humidified 37° C. CO₂ incubator for a specified period of time. During this incubation period, the immobilized antibody, in the immediate vicinity of the secreting cells, binds secreted analyte. After washing away any cells and unbound substances, a biotinylated polyclonal antibody specific for the chosen analyte is added to the wells. Following a wash to remove any unbound biotinylated antibody, alkaline-phosphatase conjugated to streptavidin is added. Unbound enzyme is subsequently removed by washing and a substrate solution (BCIP/NBT) is added. A blue-black colored precipitate forms and appears as spots at the sites of cytokine localization, with each individual spot representing an individual analyte-secreting cell. The spots can be counted with an automated ELISPOT reader system or manually, using a stereomicroscope.

Patient Population

Ninety five healthy volunteers (HV) were recruited and samples were collected under a study reviewed and approved London—Surrey Borders National Research Ethics Committee, project number 12/LO/0105 (London, UK). All subjects gave written informed consent before the donation of samples. All individuals were HLA-typed at HLA class I A and B loci by SSP-PCR.

JCPyV and BKPyV Serology

The humoral response to JCPyV was assessed by testing the serum for presence of antibodies against recombinant viral protein 1 (VP1). Human serum samples were collected and stored at −80° C. until use. Recombinant JCPyV VP1 major capsid protein (Abcam, ab74569, Cambridge, UK) was diluted to 1 μg/ml in Dulbecco's Phosphate buffered saline (DPBS; Gibco, Gent, Belgium) and used for coating ELISA plates (C96 Maxisorp plates, Nunc, Erembodegem, Belgium) overnight at room temperature (RT). Plates were washed once with DPBS containing 0.05% Tween-20 and blocked for >1 h with 1% bovine serum albumin (BSA) in DPBS. Serum samples were diluted 1:200 in 1% casein in PBS (Blocker™ Casein, Thermo Scientific) and incubated for 1 h at RT (100 μl/well) after removal of blocking agent. After washing four times, plates were incubated for 30 min at RT with a Horse Radish Peroxidase (HRP)-labeled secondary Ab mouse monoclonal anti-human IgG-HRP (OrthoClinical Diagnostics); diluted 1:1000 in Blocker™ Casein. Following final washing (four times) signals were developed by adding 100 μl/well SureBlue™ TMB Microwell Peroxidase substrate (KPL) for 10 min in the dark. Reaction was stopped by adding an equal volume of 1N HCl and absorbance was read at 450 nm.

To determine the presence of anti-BKPyV antibodies in human serum samples the same protocol was applied as described for JCPyV, but in this case ELISA plates were coated with recombinant BKPyV VP1 protein produced in and purified from a baculovirus expression system (Eurogentec, Liège, Belgium).

For both JCPy virus serology and BKPy virus serology we defined the log₂(ODₜₑₛₜ sample/OD_{control}). A value below 1 was classified as seronegative, equal to or above 1 as seropositive.

CTL Epitope Prediction for JCPyV and BKPyV Reference Strains

JCPyV MAD-1 strain (acc. Number: J02227) and BKPyV Dunlop strain (acc. Number: V01108) sequences were subjected to immunoprofiling using the Epibase platform's “HLA class I 9+10-mers-global v2.2” settings (Lonza, Cambridge, UK). All sequences were scanned for the presence of putative HLA class I restricted epitopes. Profiling was performed at the allotype level for 16 A and 12 B types. To identify epitopes specific to JCPyV proteins, a filter derived from BKPyV protein sequences was applied to JCPyV sequences. Similarly, for identifying BKPyV-specific epitopes, a filter derived from JCPyV protein sequences was applied to the BKPyV sequences. Also, a filter derived from human proteome sequences was applied to the 10 proteins to eliminate analogy with self-peptides.

Reagents

The 9-mer and 10-mer JCPy virus test peptides were synthesized and HPLC purified to >90% (Thermo Fisher Scientific, GmbH). Peptides were solubilized in DMSO and used in ELISPOT (Mabtech, Sweden) at 10 μM.

JCPyV VP1 and BKPyV VP1 PepTivator pools (Miltenyi Biotec, Bergisch Gladbach, Germany) contain 15-mer peptides overlapping by 11 amino acids across the entire VP1 protein. The working concentration for each peptide was 1 μg/ml. CEF control peptide pool (Anaspec, Calif., US) was used as positive control during the experiments at 2 μg/ml.

The peptide pool of top 10 peptides originating from the initial screening (JCPyV pool) was solubilized in DMSO at 12.5 mg/ml per peptide with a final working concentration of the pool around 10 μM.

Standard assay medium is RPMI 1640 completed with 10% FBS (Lonza, Verviers) and the serum-free medium used was X-vivo 15 medium (Lonza, Verviers).

PBMC Isolation, Freezing, Thawing

Peripheral blood mononuclear cells (PBMC) from the healthy subjects were prepared from whole blood within 6 hours of blood withdrawal. PBMC preparations were frozen using an automated controlled-rate freezing machine and cryopreserved in vapor phase nitrogen. The quality of each PBMC preparation was analyzed using a short term polyclonal T cell activation assay and 7 day activation with keyhole limpet hemocyanin (KLH) or tetanus toxoid (TT). Acceptance criteria for PBMC used in screening assay is set on viability>85% and a positive functional T cell response (measured by short-term polyclonal stimulation).

For some experiments, an overnight rest period for the cells was included. PBMC samples were thawed, washed and incubated overnight (16-20 hours) in RPMI 10% FBS in a humidified atmosphere at 37° C. and 5% CO2. CD8+ T cell enrichment was carried out post-rest period and immediately prior to antigen stimulation.

CD8+ T Cell Enrichment

To enrich for the CD8+ T cell population, CD4+ cells were depleted from the PBMC using magnetic cell separation, according to the manufacturer's instructions (StemCell Technologies, Vancouver, Canada).

Direct Ex Vivo IFN-γ ELISPOT Assay

PBMC from selected subjects were thawed, washed and seeded onto anti-IFN-γ coated 96-well plates (Mabtech, Sweden) at 2.5*10⁵ or 5*10⁵ cells per well. Test antigens were diluted in the assay media and added to the cells at the specified doses. Assay medium containing DMSO, and assay medium alone were included as blank conditions for all donors. Cells were incubated for 16-18 hours in a humidified atmosphere at 37° C., 5% CO2. After the incubation period the cells were washed from the plates and an in-house spot development procedure was used to determine IFN-γ production (Lonza, Cambridge, UK). Upon completion of spot development, the plates were scanned, counted and QC analysis conducted using the ImmunoSpot Analyzer (Cellular Technologies Ltd, Cleveland, US).

Statistical Analysis

The spot count per well (i.e. the number of IFN-γ secreting cells per well) in response to each test antigen was analyzed. Mean values were calculated from the triplicate wells for each test condition. Data analysis was then carried out using the distribution free resampling method (DFR). The DFR method compares each test condition against the blank for each donor and calculates whether the difference is statistically significant (p<0.1).

Direct Ex Vivo IFN-Gamma ELISPOT Optimization

Previously identified HLA class I T cell epitopes were used as test antigens for the optimization of the IFN-gamma T cell responses by ELISPOT, namely VP1 p36 and p100, which are described as HLA-A*02 epitopes. Therefore CTL responses to JCPyV VP1p36 and p100 in total PBMC were compared to CD8+-enriched PBMC in 3 HLA-A*02 subjects and one non-HLA-A*02 subject (donor 49). As positive controls, CEF peptide pool and JCPyV and BKPyV VP1 15-mer peptide pools were included. As shown in FIG. 1, while higher numbers of total PBMC per well (5*10⁵ versus 2.5*10⁵) increased the number of detectable spots in test antigens, there was also a corresponding increase in spots in the blank condition, thereby not resulting in an enhanced detection of significant responses. However, when CD8+-enriched PBMC were used (5*10⁵ cells per well; condition C) the number of IFN-γ secreting cells in response to test antigens was increased without an increase in background spots. All four subjects showed a significant response to both BKPyV and JCPyV VP1 peptide pools. One subject responded significantly to VP1 p36 and two subjects responded to JCPyV VP1p100.

It has been reported that for some viral peptides, the use of serum-free assay medium and overnight resting of the PBMC before stimulation with peptide can increase peptide specific T cell responses. The influence of assay medium and overnight resting of PBMC was evaluated using only CD8+-enriched PBMC from 5 HLA-A*02 subjects and one non-HLA-A*02 subject. As shown in FIG. 2, PBMC enriched for CD8+T-cells with the standard ELISPOT assay medium without overnight resting period (condition D) had the best sensitivity. All six donors responded to the CEF peptide pool and four of the donors responded significantly to JCPyV VP1 peptide pool. Of these four donors, three of them also responded to JCPyV VP1 p100 peptide and one of them to JCPyV VP1 p36.

The ELISPOT with CD8+-enriched PBMC (5*10⁵ cells per well) with the standard ELISPOT assay medium without overnight resting period was used in further experiments.

Epibase in Silico Prediction and ELISPOT Screening of JCPyV MHC Class I Peptides

All viral protein (VP1, VP2, agnoprotein, common T, large T Antigen (LTAg), and small t Antigen (stAg) from both JCPyV and BKPyV) sequences were scanned for the presence of putative HLA class I restricted epitopes. Profiling is performed at the allotype level for 16A and 12B, i.e. 28 HLA class I receptors in total. As an example of the epitope prediction, FIG. 3 shows a heatmap of results obtained for JCPyV agnoprotein. The heat map clearly shows that there are many more predicted epitopes than can be feasibly tested. Therefore, during the further selection of epitopes with high expected immunogenicity potential, the following aspects were considered: high HLA-A and -B impact of epitopes, high promiscuity of epitopes, specificity to JCPyV proteins, potential preference for 9-mer peptides, minimal similarity of epitopes to human proteome sequences, location of epitopes in the JCPyV protein sequences Based on the in silico predictions, 98 peptides of the JCPyV proteins were finally selected, representing potential T cell epitopes for the major HLA class I allotypes in the global population. To test immunogenicity of these 98 peptides the ten most common HLA allotypes were selected, 5 HLA-A and 5 HLA-B allotypes (A*0101, A*0201, A*0301, A*1101, A*2402, B*0702, B*0801, B*1501, B*3501 and B*4001) and for each HLA allotype we aimed to have approximately 10 peptides that gave the strongest signal in Epibase. Alongside, the positive controls (CEF, VP1 peptide pool, and VP1 p100) and negative controls (medium blank and DMSO) were tested. The control peptide pools gave responses in the expected range, in 87% and 60% of all subjects tested, showing a positive response to the CEF and JCPyV VP1 peptide pools, respectively. As an example, FIG. 3 shows an overview from the predicted peptides, the tested peptides, and the peptides that induced an immune response for agnoprotein for 9-mer and 10-mer peptides. The results for the other proteins can be found in supplementary Table 1 and 2. A total of 50/98 peptides did not result in any significant stimulation of a T cell response, another 30 peptides gave a response in less than 20% of the tested donors; there were 8 peptides that provoked a response in 20 to 30% of the tested donors; and finally, 10 peptides (9 new peptides and the already known JCPyV VP1 p100) could be considered as immunodominant, since they gave a significant immune response in more than 30% of the tested individuals.

A total of 44 out of 92 (48%) subjects tested showed a positive response to the JCPyV VP1 p100 9-mer peptide with the A*0201 and A*2402 allotypes showing the highest number (58%) of significant responses as predicted by Epibase™ in silico (FIG. 4). It may be difficult to define if the responses to the JCPyV VP1 p100 9-mer peptide are specific memory T-cells to JCPyV VP1 since there is evidence of CD8 T cell cross reactivity with determinants from SVPy40. Interestingly, the direct comparison of the VP1 p100 9-mer with the VP1 p100 10-mer (AP00388) in the A*2402 donors showed that the 10-mer was far less potent than the 9-mer at inducing CD8+ T cell responses.

Based on the number of responding subjects, the top-10 peptides identified are summarized in Table 1. The peptide most recognized (in 90% of the subjects) was LTAg389 (MAGVAWIHCL). There were 7 peptides that showed a significant response (p<0.01) in at least 3 of the allotype-specific subjects tested. These 7 peptides cover 6 of the 10 HLA class I allotypes assessed in this study. The screening also identified an additional 3 peptides covering an extra 2 HLA class I allotypes in at least 3 subjects. In total, these 10 peptides represent CD8 T cell epitopes from all 5 JCPyV proteins and cover 8 of the 10 HLA allotypes tested (Table 1). No significant immune responses against the predicted peptides were identified for the B*0702 or B*3501 allotypes. In conclusion, the direct ex vivo IFN-γ ELISPOT assay was able to identify a number of peptides that were predicted in silico to represent CD8+ T cell epitopes found in JCPyV. Peptides were identified in all 5 JCPyV proteins and covered 8 of the 10 HLA allotypes tested in this project.

Validation of the Newly Identified Immunodominant Epitopes

To verify whether the newly identified epitopes were really immunodominant, the 10 newly identified peptides were tested individually or as a peptide pool in the optimized CD8+ T cell enriched direct ex vivo ELISPOT assay. Twenty subjects were selected which cover 8 of the 10 HLA allotypes that were initially selected. HLA-B*0702 and HLA-B*3501 were excluded because no positive peptides were identified for these allotypes in the previous screens. The pool of the 10 newly identified epitopes was tested alongside the 10 individual peptides. As shown in Table 2, all peptides tested showed strong capacity for inducing a T cell response in the 20 selected donors with 70-100% of donors responding to each peptide. AP00386 (stAg₁₂₅) and AP00457 (VP1 68) induced the highest number of T cell responses with 100% and 90% of the donors responding respectively. The peptide pool was also very potent and induced a T cell response in 100% of donors tested. In conclusion, the novel JCPyV CD8+ T cell epitopes identified in silico induced a robust response in an optimized direct ex vivo IFN-gamma ELISPOT assay.

Correlation Between Antibodies Against VP1 and T-Cell Responses.

It has been shown that antibody seropositivity for JCPy virus (against recombinant VP1) does not necessarily correlate with presence of memory T cells. Therefore also JCPyV-specific humoral responses were measured in serum using an ELISA set up. Serum samples were available from all 92 donors of which 39% turned out to be seronegative for anti-JCPyV VP1 antibodies. As shown in FIG. 5: 22 of these seronegative individuals (log₂(OD/control)<1) had a significant (adj p<0.1) CTL immune response to one or more of the 10 selected JCPyV epitopes. In Table 3, the individual haplotypes and reactive peptides for these 22 individuals are summarized. Almost all of these seronegative subjects (82%) reacted against VP1 p100 independently of the HLA-type, a result comparable to the results obtained for this peptide in the global population. The other peptide that was recognized in 8 out of 22 (36%) was agnoprotein 27 (RILIFLLEFL).

The subjects who were seronegative for JCPy virus were tested for the presence of antibodies against BKPyV recombinant VP1. They tested all seropositive for anti-BKPyV VP1 antibodies (log2(Signal/control)>1) except for 1 donor (Donor 37). Since immunological cross reactivity between JCPyV and BKPyV has been demonstrated previously, a homology overview for the most important epitopes between JCPyV and BKPyV was prepared (Table 4). Most of the epitopes tested for JCPy virus show an important homology with BKPy virus, with only a few amino acids that are different.

Part of the current invention is the method to use a direct ex vivo ELISPOT assay to measure cellular immune responses to JCPyV. The main focus was on CD8+ T-cells as these are the key effectors involved in killing virus-infected host cells when viral epitopes are presented on the cell surface via MHC class I. Computer algorithms were used to predict peptide binding to HLA-A and B molecules and led to identification of hundreds of predicted epitopes with median or strong prediction. 98 peptides were selected for screening in healthy subjects, resulting into the identification of 9 new epitopes that were at least recognized by 3 different donors. The two epitopes most frequently recognized were found in the T antigens: large T (LT)Ag₃₈₉ (90% of donors) and small t (st)Ag₁₂₅ (80% of donors).

Effector memory T-cells rapidly release cytokines such as IFN-γ when re-exposed to antigen. Previous studies using ELISPOT examined IFN-γ expression in T cells following long-term activation with JCPyV peptides for up to 14 days. Prolonged activation allows long-term quiescent T cell memory to be reactivated and to expand, meaning that a positive response does not necessarily signify an ongoing immune response. In the present inventive assay, effector memory T-cells were measured directly ex vivo, which leads to immune responses that are lower in magnitude. This can be explained by the pattern of viral replication and the anatomical sites of viral latency. In most individuals, primary infection happens during childhood and the virus subsequently remains latent or replicates in the kidney or the lymphoid organs. Low-level antigen synthesis or inefficient antigen presentation at these sites may lead to limited CD8 T-cell responses. However, the broad specificity of the CD8 T cell responses measured here indicated that multiple JCPyV peptides can be recognized by Caucasian individuals, and this is consistent with the fact that JCPyV is a highly prevalent virus to which the human immune system is well adapted.

Previously, two CD8 T cell epitopes, peptides VP1 p100 and VP1 p36 were described as immunodominant in HLA-A*0201-restricted donors. Here the VP1 p100 was represented by peptide AP00410, the VP1 peptide p36 was only used to optimize the assay. Epibase™ In silico predictions showed that JCPyV VP1 p36 peptide binds strongly to HLA-A*0202 but not to HLA-A*0201. Interestingly, the single donor that responded to JCPyV VP1 p36 peptide had the HLA-A*0202 allele (FIG. 2). This might explain why only in this donor a significant response was measured. 37% of the donors tested showed a positive response to JCPyV VP1 p100 9-mer peptide (AP00410), independent of HLA-type. A*0201 and A*2402 allotypes showed the highest number of significant donor responses as predicted by Epibase™ in silico. It may be difficult to define if the responses to JCPyV VP1 p100 9-mer peptide were specific memory T-cell responses to JCPyV VP1 since there is evidence of CD8+ T cell cross reactivity with determinants of SVPy40.

Besides VP1 p100 two peptides were found that were even more frequently recognized, namely LTAg₃₈₉ (MAGVAWIHCL), followed by stAg₁₂₅ (FLRSSPLVWI). These epitopes have not been described earlier. The epitopes most frequently recognized were localized in VP1 and large T-antigen. VP1 encodes the major capsid protein of JCPyV. Large T antigen encodes a multifunctional protein that regulates transcription of the structural genes. They also play different roles in virus replication. In the early stage the JCPy virus attaches the host cells by receptors containing sialic acid. The VP1 capsid protein is responsible for receptor binding. VP2 and VP3 capsid protein facilitate entry and uncoating. The virions are taken up by receptor mediated endocytosis and are transported to the nucleus. They enter the nucleus through the nuclear pores and uncoat inside. The virus cellular histones are transcribed by host RNA polymerase II into early mRNAs which are translated into the early antigen protein. In the late stage the viral DNA is replicated with enzymes produced by the T antigens. The DNA is then translated to produce late mRNAs which are translated into capsid proteins. This implicates that subjects that had induced a response against VP1 could have been exposed to or infected by the virus, while subjects that had induced a response against T antigens have been infected with the virus, because this antigen is only present when real virus production had taken place. Another epitope that was frequently recognized was localized in the agnoprotein: agno27 (RILIFLLEFL).

The 10 peptides that showed a significant immune response in at least 3 different donors were tested in 20 donors, separately and as a pool, to validate their immunodominance. Each individual peptide induced a significant T cell response in at least 14 of the 20 donors tested, with stAg125 (FLRSSPLVWI) inducing a response in all 20 donors tested. The peptide pool also induced a significant T cell response in all 20 donors tested.

The VP1 p100 is the known immunodominant epitope for HLA-A*0201 found in healthy volunteers and appears to correlate with protection from PML in HIV infected individuals. To investigate whether the newly identified epitopes could prevent seropositivity for JCPy virus in healthy volunteers, JCPy serology was measured and correlated with the presence of CTL epitopes. Seropositivity for JCPy virus did not correlate with the presence of effector memory T-cells, which is related to virus reactivity. In the group of healthy subjects tested that induced a significant immune response against JC virus epitopes, 39% were sero-negative for JCPyV. Among the seronegative individuals (n=22) almost all responded to the known immunodominant epitope VP1p100, but also a lot of them (8 out of 22) reacted against agno27 peptide, indicating that in this study in 39% of healthy individuals, induction of cellular immune responses against certain JCPy virus epitopes could prevent seropositivity to JCPy virus. However, these data should be interpreted with some cautions. Primarily, it cannot be excluded, that parts of these are false-negatives, as it was recently stated that the false-negative rate of the JCPyV serology can be as high as 37%. This indicates that JCPyV sero-status does not appear to identify all patients infected with JCPyV. Secondly, there can be some cross reactivity with BKPy virus, VP1 p36 and VP1 p100, show cross reactivity with BKPy virus namely BKV VP1 p44 and BKV VP1 p108 and also one other epitope in LTAgₚ₂₇ (LPLMRKAYL) was described earlier to show this. BKPy virus is a human polyomavirus that has 75% sequence homology with JCPyV and causes nephropathy in kidney transplant recipients. Therefore, BKPy virus serology were measured in the individuals who were seronegative for JCPyV. Only one subject (donor 37) was seronegative for BKPy virus. This individual donor 37 was extremely interesting, because he/she was seronegative for both JCPy and BKPy virus but presented significant immune responses against JCPy virus indicating that this person is protected against JCPy virus infection. Most of the epitopes tested for JCPy virus show an important homology with BKPy virus, with only a few amino acids that are different. These findings suggest that JCPyV-seronegative individuals, but seropositive for BKPyV, can induce a CTL response against JCPy epitopes, hence limited cross-reactivity between both viruses at the cellular immunity cannot be excluded. Most of the epitopes (65%) for JCPy virus that were predicted in silico and that induced an immune response were not at all predicted for BKPy virus.

In conclusion, the direct ex vivo ELISPOT assay according to the invention has been able to identify a number of peptides that were predicted in silico to represent CD8+ T cell epitopes found in JCPyV. Peptides were identified in all 5 JCPyV proteins and in different HLA-allotypes. It is anticipated that responses against these epitopes can be correlated with protection against PML.

### FIGURE LEGENDS

FIG. 1: Impact of Cell Amount and CD8+ T Cell Enrichment on JCPyV Peptide Induced IFN-Gamma Responses.

The figure shows the number of IFN-gamma secreting cells in response to the DMSO, blank and test antigens. (A) PBMC 2, 5*10⁵ per well, (B) 5*10⁵ per well (C) CD8-enriched cells 5*10⁵ per well. Starred bars (*) indicate a significant response as determined by DFR method.

FIG. 2: Impact of Assay Medium and Overnight Resting on JCPyV Peptide Induced IFN-Gamma Responses.

The number of IFN-gamma secreting cells in 5*10⁵ CD8-enriched cells/well in response to DMSO, blank and test antigens varied according to the following: (A) serum free medium with overnight rest period, (B) serum-free medium with no overnight rest period, (C) serum containing medium with an overnight rest period and (D) serum containing medium with no overnight rest period.

FIG. 3. Heatmap Predicted and Tested Responses to JCPyV Peptide

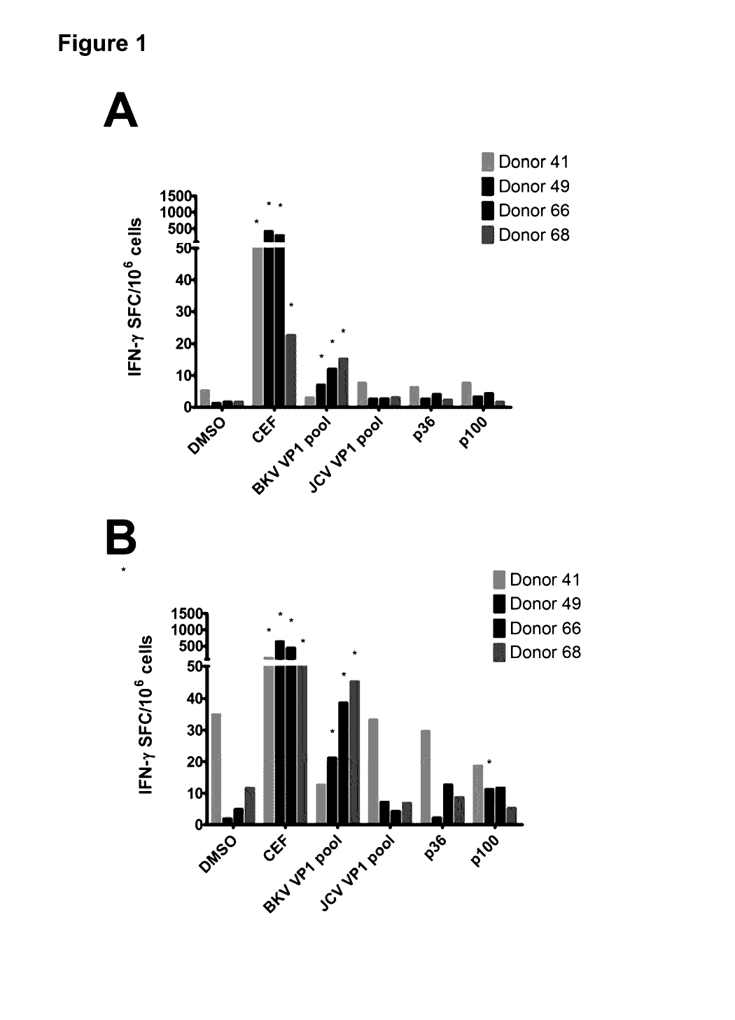

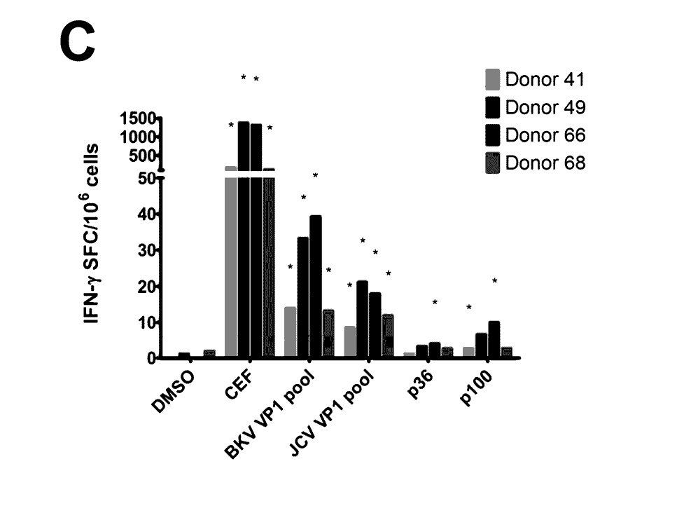

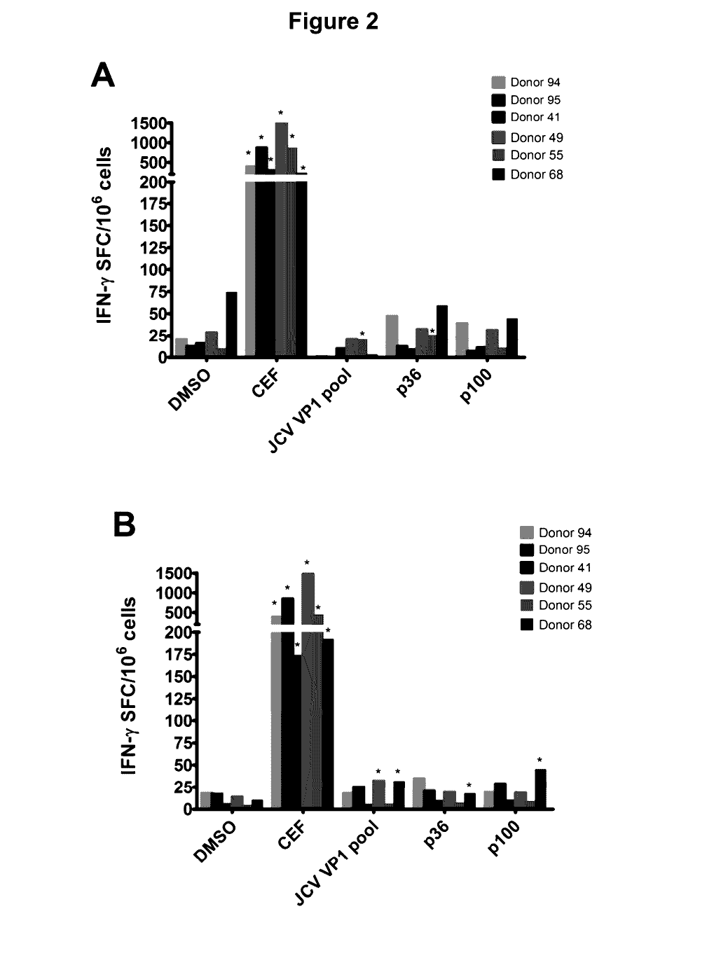

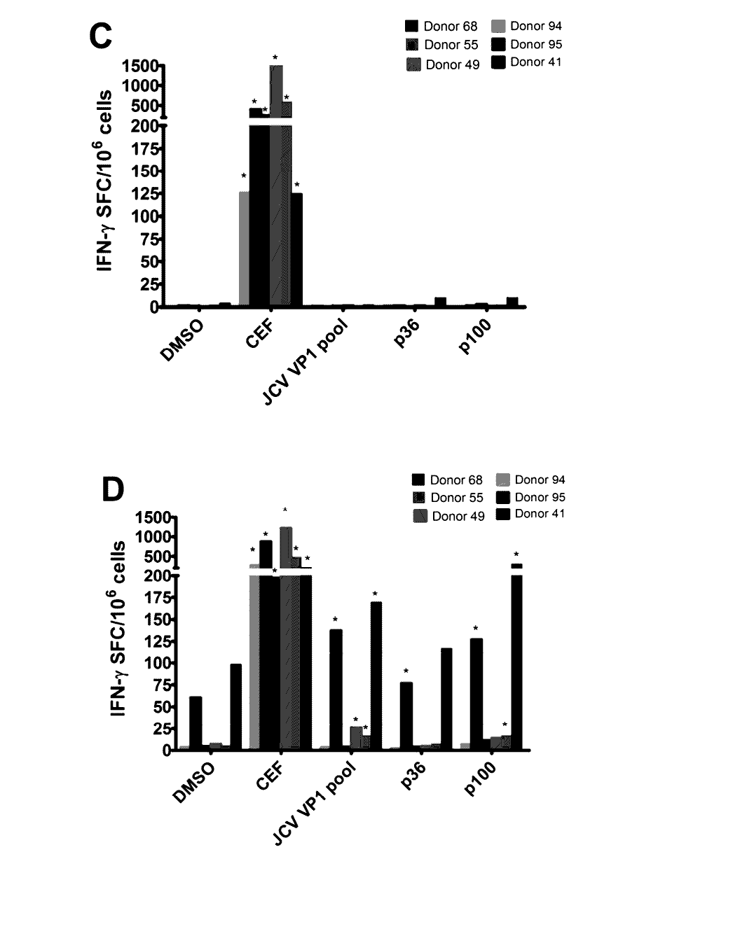

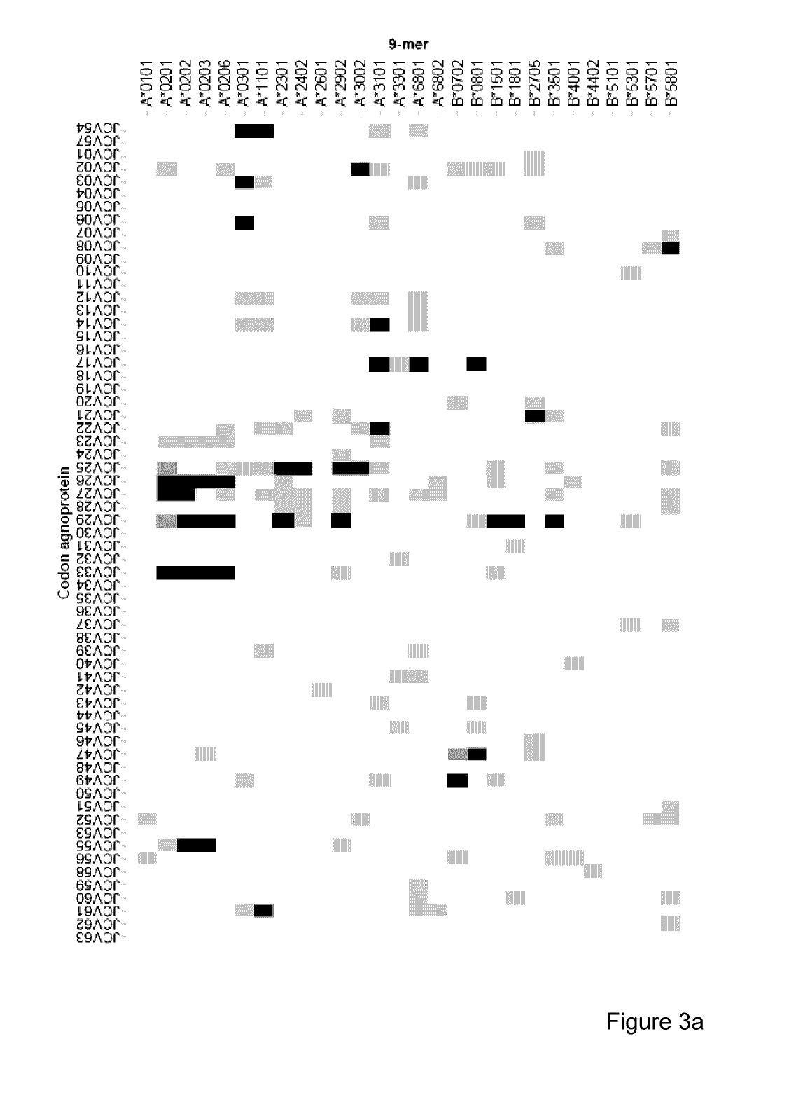

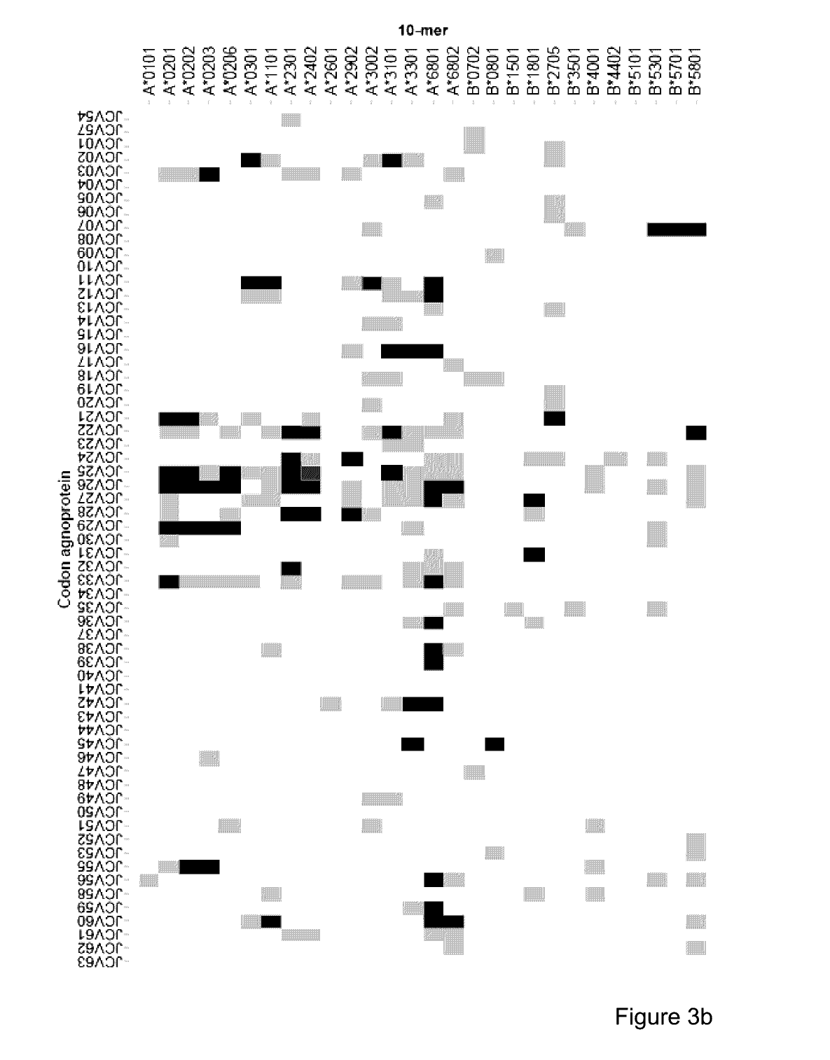

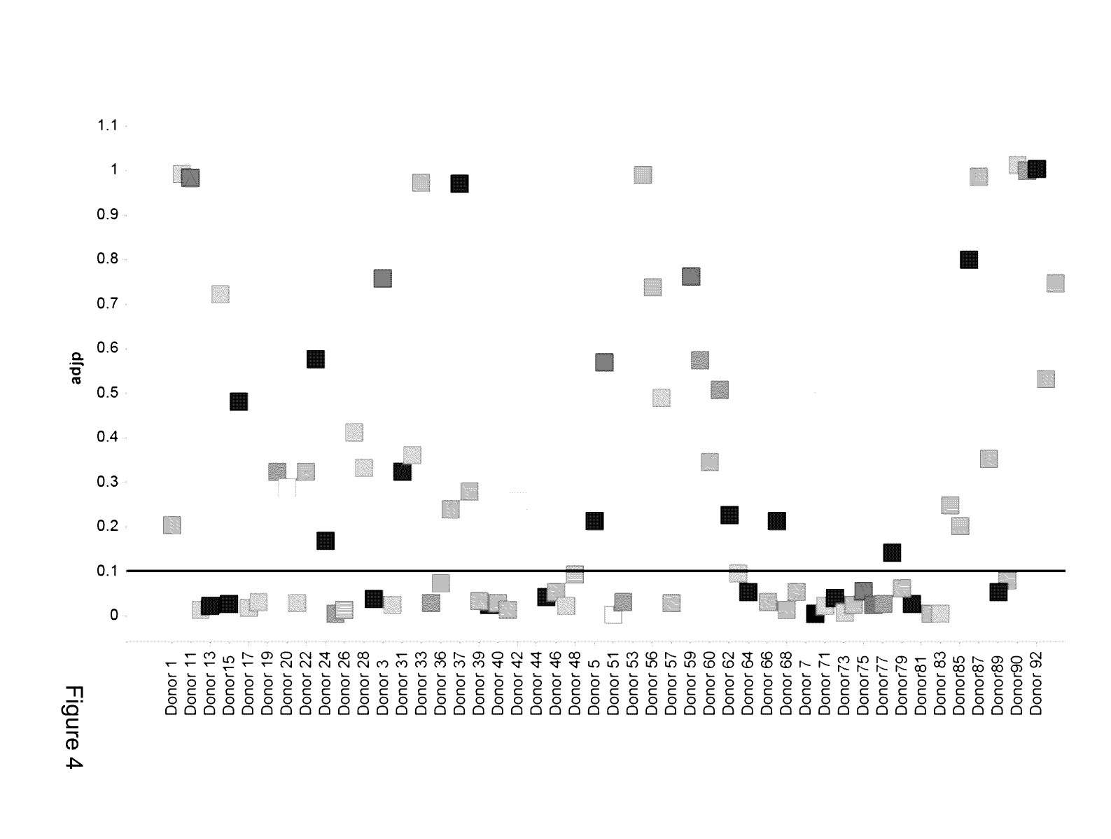

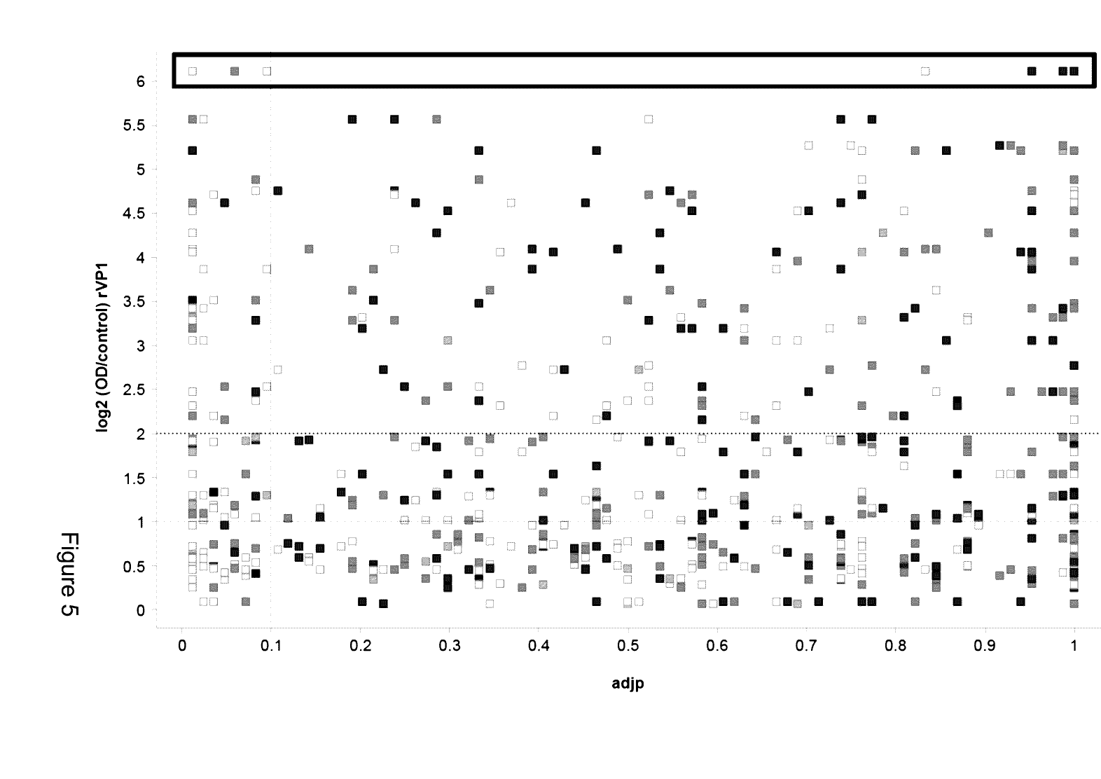

All 9-mer and 10-mer predicted peptides of agnoprotein are represented in this figure. With the epibase™ in silico tool it was investigated whether they were predicted epitopes for the major HLA class I allotypes in the global population. Grey (not predicted epitopes), green (predicted epitopes that are medium binders), blue (predicted epitopes that are strong binders) The 10 peptides for each of the HLA-allotypes that gave the highest prediction (strong binders) were eventually tested in 10 donors per HLA-allotype:, Orange (response in 1 donor), pink (response in all donors). On the Y-axis the codon of the epitope from agnoprotein is mentioned.

FIG. 4: Immune Responses Against JCPyV VP1 p100 9-mer Peptide

Responses are shown against the JCPyV VP1 p100 9-mer. The x-axis represent the donors tested, the Y-axis the p-value from the ELISPOT assay. p-values below 0.1 are considered significant. The donors are colored by the HLA-A type. Green are the donors with HLA-A*2402 allotype and pink are the ones with HLA-A*0201 allotype. The other colors represent the other HLA-A allotypes selected in this study.

FIG. 5: Correlation Plot Between Antibodies Against Recombinant VP1 and T-Cell Responses

Serum samples from all individuals were tested for humoral responses against recombinant JCPyV VP1 protein. Obtained signals are plotted on the y-axis as log2 (OD/control). In the x-axis the significance of the induced responses against peptides is plotted. The color represents the different proteins (pink=agnoprotein, grey=common T antigen, blue=T-antigen, green=t-antigen, yellow=VP1, orange=VP2). The horizontal line at 1 on the Y-axis is the difference between seronegative subjects (<1) and seropositive subjects (>1). The vertical line at 0.1 on the x-axis indicate the difference between significant induced responses (<0.1) and not significant responses (>0.1). In the boxed area at the top of the figure: as an example, an individual who is seropositive for JCPy VP1 was tested in a T cell assay against 10 peptides, for 3 peptides a significant IFN-gamma release was measured (adjp<0.1). Only responses against 7 peptides are visible, 3 peptides are hidden under the others.

TABLE 1
Summary of peptides tested that gave
a significant response in
at least 3 different donors

| | | | | | Posi- | |
|---|---|---|---|---|---|---|
| | | | | | tive | Pre- |
| | | JCPyV | | | Donors | dicted |
| Rank- | | pro- | Posi- | | (p < | HLA- |
| ing* | Peptide | tein | tion | Sequence | 0.1) | type |
| 1 | AP00423 | large | 389 | MAGVAWIHCL | 9 | B*0801 |
| | | Tant | | | | |
| 2 | AP00386 | small | 125 | FLRSSPLVWI | 8 | A*0301 |
| | | Tant | | | | |
| 3 | AP00410 | VP1 | 100 | ILMWEAVTL | 7 | A*2402 |
| 4 | AP00400 | VP2 | 159 | STISQAFWNL | 6 | A*1101 |
| 5 | AP00402 | agno | 27 | RILIFLLEFL | 6 | A*2402 |
| | | pro- | | | | |
| | | tein | | | | |
| 6 | AP00457 | VP1 | 68 | FESDSPNRDM | 6 | B*4001 |
| 7 | AP00377 | VP1 | 292 | YPISFLLTDL | 5 | A*0201 |
| 8 | AP00441 | VP2 | 92 | SAIAQLGYRF | 5 | B*1501 |
| 9 | AP00367 | VP1 | 95 | LTCGNILMW | 4 | A*0101 |
| 10 | AP00460 | VP2 | 32 | GEAAATIEV | 3 | B*4001 |

*rank based on number of responding donors.

TABLE 2
Summary of the individual donor responses to the top 10 peptides

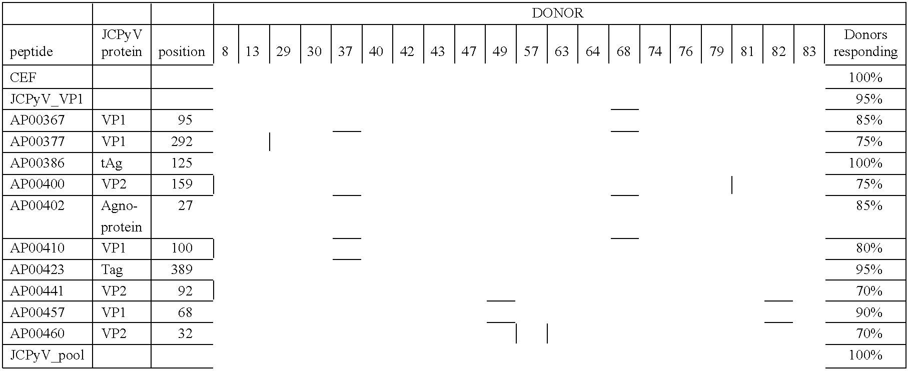

TABLE 3
Overview of specific immune responses (adjp < 0.1) in
serologic negative (=Log2(OD/control) < 1)

| | | | Protein start codon(peptide length) | | | | |
|---|---|---|---|---|---|---|---|
| Donor | HLA-A | HLA-B | LTAg | tAg | VP1 | VP2 | agno |
| Donor 9 | A*0201\|A*0301 | B*3501\|B*4501 | 269(9) | 98(10) | 100(9) | 120(9) | |
| | | | | | 165(9) | | |
| Donor 17 | A*0101\|A*2402 | B*0702\|B*0801 | 417(9) | | 100(9) | | |
| | | | 389(10) | | | | |
| Donor 28 | A*1101\|A*2402 | B*1301\|B*5201 | | | | 159(10) | |
| Donor 35 | A*0301\|A*0301 | B*0702\|B*0702 | 139(9) | | 100(9) | | |
| | | | | | 306(9) | | |
| Donor 37 | A*0201\|A*0301 | B*1501\|B*4402 | | 125(10) | | | |
| Donor 42 | A*0201\|A*0201 | B*4402\|B*4901 | | | 100(9) | 195(10) | 27(10) |
| | | | | | 292(10) | | |
| Donor 46 | A*0201\|A*0301 | B*0702\|B*2705 | 608(10) | 125(10) | 100(9) | | |
| | | | | | 100(10) | | |
| Donor 47 | A*2402\|A*3101 | B*0702\|B*0705 | 368(10) | | 100(9) | | 27(10) |
| | | | 189(9) | | | | |
| | | | 288(9) | | | | |
| Donor 49 | A*0101\|A*0101 | B*0702\|B*0801 | 389(10) | | 100(9)*2 | | |
| Donor 52 | A*0301\|A*2601 | B*0702\|B*3801 | | | 100(9) | | |
| Donor 53 | A*2402\|A*6801 | B*1501\|B*4001 | | | 100(9)*2 | 32(9) | |
| | | | | | 68(10) | | |
| Donor 63 | A*0301\|A*2402 | B*0702\|B*4001 | | | 100(9) | 32(9) | |
| | | | | | 68(10 | 40(9) | |
| Donor 64 | A*0101\|A*1101 | B*5201\|B*5501 | | | 100(9)*2 | 159(10) | |
| | | | | | 95(9) | | |
| Donor 68 | A*0201\|A*0201 | B*1302\|B*4402 | 361(9) | | 100(9) | | 27(10) |
| | | | | | 292(10) | | |
| Donor 69 | A*0201\|A*3301 | B*1402\|B*5101 | | | 100(9) | | 27(10) |
| Donor 70 | A*3201\|A*3303 | B*0702\|B*0702 | 486(10) | | 100(9) | | |
| Donor 71 | A*0101\|A*2402 | B*3801\|B*4404 | | | 100(9) | | 27(10) |
| Donor 73 | A*2402\|A*3101 | B*4402\|B*5401 | | | 100(9) | | 27(10) |
| Donor 74 | A*0201\|A*2601 | B*4601\|B*6701 | | | 100(9) | | 27(10) |
| | | | | | 292(10) | | |
| Donor 78 | A*0101\|A*0101 | B*0702\|B*5701 | 13(9) | | 95(9) | | |
| Donor 80 | A*0201\|A*3001 | B*1801\|B*4201 | | | | | 27(10) |
| Donor 87 | A*0101\|A*0201 | B*0702\|B*5701 | 27(9) | | | | |

TABLE 4
Homology between BK and JC virus epitopes

| | | | | | Predicted | Prediction | Prediction |
|---|---|---|---|---|---|---|---|
| | | | sequence | sequence | HLA- | JC | BK |
| Protein | codon | length | JC | BK | type^{a} | virus^{b} | virus^{c} |
| agno- | 27 | 10 | RILIFLLEFL | RIFIFILEL | A* | strong | strong |
| protein | | | | | 0201 | | |
| largeTant | 139 | 9 | FPVDLHAFL | FPSDLHQFL | B* | strong | Not |
| | | | | | 0702 | | predicted |
| largeTant | 189 | 9 | GFGGHNILF | MCAGHNIIF | A* | strong | Not |
| | | | | | 2402 | | predicted |
| largeTant | 269 | 9 | VSWKLVTQY | VSWKLITEY | B* | strong | Not |
| | | | | | 3501 | | predicted |
| largeTant | 288 | 9 | LLMGMYLDF | LLLGMYLEF | A* | strong | Not |
| | | | | | 2402 | | predicted |
| largeTant | 368 | 10 | FLLDKMDLIF | HILDKMDLIF | A* | strong | Not |
| | | | | | 2402 | | predicted |
| largeTant | 389 | 10 | MAGVAWIHCL | MAGVAWILCL | B* | strong | Not |
| | | | | | 0801 | | predicted |
| largeTant | 417 | 9 | IPKKRYWLF | VPKRRYWLF | B* | strong | Strong |
| | | | | | 0801 | | |
| largeTant | 608 | 10 | ISMYTFSTMK | ISMYTFSTRK | A* | strong | Not |
| | | | | | 0301 | | predicted |
| smallTant | 98 | 10 | WPNCATNPSV | WPICSKKPSV | B* | strong | Strong |
| | | | | | 3501 | | |
| smallTant | 125 | 10 | FLRSSPLVWI | FLRKEPLVWI | A* | strong | Medium |
| | | | | | 0301 | | |
| VP1 | 68 | 10 | FESDSPNRDM | FSSDSPERKM | B* | strong | Not |
| | | | | | 4001 | | predicted |
| VP1 | 95 | 9 | LTCGNILMW | LTCGNLLMW | A* | strong | Not |
| | | | | | 0101 | | predicted |
| VP1 | 100 | 9 | ILMWEAVTL | LLMWEAVTV | A* | strong | Not |
| | | | | | 2402 | | predicted |
| VP1 | 100 | 10 | ILMWEAVTLK | LLMWEAVTVQ | A* | strong | Not |
| | | | | | 0301 | | predicted |
| VP1 | 165 | 9 | YPDGTIFPK | YPDGTITPK | B* | strong | Not |
| | | | | | 3501 | | predicted |
| VP1 | 292 | 10 | YPISFLLTDL | YPISFLLSDL | A* | strong | Not |
| | | | | | 0201 | | predicted |
| VP1 | 306 | 9 | TPRVDGQPM | TQRVDGQPM | B* | strong | Not |
| | | | | | 0702 | | predicted |
| VP2 | 32 | 9 | GEAAATIEV | GEAAAAIEV | B* | strong | Strong |
| | | | | | 4001 | | |
| VP2 | 40 | 9 | VEIASLATV | VQIASLATV | B* | strong | Not |
| | | | | | 4001 | | predicted |
| VP2 | 120 | 9 | MALQLFNPE | MALELFNPD | B* | strong | Medium |
| | | | | | 3501 | | |
| VP2 | 159 | 10 | STISQAFWNL | ATISQALWHV | A* | strong | Medium |
| | | | | | 1101 | | |
| VP2 | 195 | 10 | RFLEETTWAI | RFLEETTWTI | A* | strong | strong |
| | | | | | 0201 | | |

^{a} Epitopes are predicted for JC virus for a certain HLA-allotype by an in silico
prediction tool
^{b} Prediction of epitopes for JC virus
^{c} Prediction of the same epitopes for BK virus

1. Method to predict the onset and/or development of PML in a patient by detection of JCV specific CTL's in blood of said patient using any of the ten (10) JCV amino acid sequences SEQ ID NO 1-10 separately or in combination with each other.

2. Method to predict the onset and/or development of PML in an immunosuppressed patient by detection of JCV specific CTL's in blood of said patient using any of the ten (10) JCV amino acid sequences SEQ ID NO 1-10 separately or in combination with each other.

3. Use of any of the ten (10) JCV amino acid sequences SEQ ID NO 1-10 separately or in combination with each other to detect JCV specific CTL's in blood of a patient to predict the onset and/or development of PML.

4. Use of any of the ten (10) JCV amino acid sequences SEQ ID NO 1-10 separately or in combination with each other to detect JCV specific CTL's in blood of a immunosuppressed patient to predict the onset and/or development of PML.
